SEQUENCE LISTING (2 sequences)

SEQ ID NO: 1 - DNA, 113 bp, Artificial
  synthetic oligonucleotide
tcgagagaggtcactgtgacctctgagagaggtcactgtgacctctctcagaggtcactgtgacctctgcgagaggtcactgtgacctctgcgagaggtcactgtgacctcta

SEQ ID NO: 2 - DNA, 113 bp, Artificial
  synthetic oligonucleotide
gatctagaggtcacagtgacctctcgcagaggtcacagtgacctctcgcagaggtcacagtgacctctgagagaggtcacagtgacctctctcagaggtcacagtgacctctc

(19) United States
(12) Patent Application Publication
(10) Pub. No.: US 2005/0118658 A1
(43) Pub. Date: Jun. 2, 2005

(54) Use of ERRalpha phosphorylation status as a breast cancer biomarker

(76) Inventors: Janet E. Mertz, Madison, WI (US); Eric A. Ariazi, Huntingdon Valley, PA (US); Richard J. Kraus, McFarland, WI (US)

Correspondence Address: QUARLES & BRADY LLP, 411 E. WISCONSIN AVENUE, SUITE 2040, MILWAUKEE, WI, 53202-4497, US

(21) Appl. No.: 10/942,387

(22) Filed: Sep. 16, 2004

Related U.S. Application Data
(60) Provisional application No. 60/560,350, filed on Apr. 7, 2004.
(60) Provisional application No. 60/504,045, filed on Sep. 19, 2003.

(51) Int. Cl. G01N 33/567; G01N 33/574; A61K 39/395

(52) U.S. Cl. 435/7.23; 424/155.1

(57) ABSTRACT

The inventors discovered that the ErbB2 signal transduction pathway can activate ERRα by inducing its phosphorylation. Based on this discovery, the present invention provides methods for determining whether a breast cancer patient is likely to respond to hormonal-blockade therapy or ErbB2-based therapy, methods for determining prognosis of breast cancer patients, methods for treating breast cancer, and methods for identifying agents that can modulate ERRα phosphorylation.

### CROSS-REFERENCE TO RELATED APPLICATIONS

This application claims the benefit of U.S. provisional application Ser. No. 60/560,350, filed on Apr. 7, 2004 and U.S. provisional application Ser. No. 60/504,045, filed on Sep. 19, 2003, both of which are herein incorporated by reference in their entirety.

### STATEMENT REGARDING FEDERALLY SPONSORED RESEARCH OR DEVELOPMENT

This invention was made with United States government support awarded by the following agency: NIH, Grant No. CA22443. The United States government has certain rights in this invention.

### BACKGROUND OF THE INVENTION

One out of eight women in the U.S. will develop breast cancer in her lifetime. Currently, estrogen receptor α (ERα), progesterone receptor (PgR) and ErbB2 (also termed HER2), are used clinically as biomarkers for breast cancer. ERα and ErbB2 are also direct or indirect treatment targets in hormonal-blockade therapies and ErbB2-based therapies for certain types of breast cancer. Recently, estrogen-related receptor α (ERRα ) has been identified as another biomarker and treatment target for breast cancer.

The steroid nuclear receptor (NR) estrogen receptor α (ERα, officially termed NR3A1 [1]) is pivotally involved in the etiology of breast cancer. ERα mediates the effects of estrogens on transcription and is expressed at high levels in approximately three-fourths of human breast tumors. It thereby serves as a critical biomarker of clinical course and target for therapy [2]. The orphan NRs estrogen-related receptor α (ERRα ; NR3B1), ERRβ (NR3B2), and ERRγ (NR3B3) [1] exhibit a high degree of sequence similarity with ERα [3]. They share multiple biochemical activities including binding to estrogen response elements (EREs). However, ERRs do not bind naturally occurring estrogens, nor has any naturally occurring ligand been identified for ERRs [3].

ERRα has been implicated to play important roles in breast cancer by modulating or substituting for ERα's activities, especially in ErbB2-positive or ERα -negative tumors. ERRα mRNA levels are similar or greater than ERα mRNA levels in approximately one-fourth of unselected human breast cancers, with the highest levels being found in tumors lacking functional ERα [4]. Furthermore, ERRα mRNA levels correlate in breast cancers with those of ErbB2 (also called HER2) [4], a marker of tumor aggressiveness. ErbB2 overexpression is associated with the ER-negative/PgR-negative phenotype in human breast cancer and thus indicates poor prognosis and insensitivity to hormonal-blockade therapy. ERRα protein expression, as exhibited by immunohistochemistry, also associates with a significantly increased risk of recurrence of breast carcinoma, adverse clinical outcome, and insensitivity to tamoxifen therapy [5].

Whereas ERα usually regulates gene expression in a ligand-inducible manner, ERRα is an orphan receptor that can interact with the p160 family of coactivators, including GRIP1 (glucocorticoid receptor interacting protein 1, SRC2), in the absence of exogenous ligands [6] via a C-terminal coactivator-binding motif [7]. Bulky amino acid side chains in ERRα's putative ligand-binding pocket, including Phe329, recapitulate interactions analogous to ones provided by ligands, thereby promoting binding of co-activators [8].

ERRα has been shown to modulate transcription of estrogen-responsive genes including lactoferrin [9], osteopontin [10], the breast cancer biomarker pS2 [11], and the estrogen-metabolizing gene aromatase [12]. The effect of ERRα binding to a transcriptional response element can be either negative or positive depending upon the specific cell type. For example, ERRα down-modulates E₂-induced transcription in ERα-positive human mammary carcinoma MCF-7 cells by an active mechanism [13], yet activates estrogen-responsive gene transcription in ERα-negative human mammary carcinoma SK-BR-3 cells [12] and a variety of other cell lines including human cervical carcinoma HeLa cells [13], human endometrial RL95-2 cells [9], human kidney HEK293 cells [10], and rat ROS 17/2.8 osteosarcoma cells [10].

The factors determining whether ERRα constitutively activates or down-modulates transcription are not known. Epidermal growth factor receptor (EGFR) and ErbB2, members of the ErbB family of transmembrane receptor tyrosine kinases, signal in part through the MEK (mitogen activated protein kinase (MAPK)) and phosphatidylinositol 3-kinase (PI3K)/Akt (protein kinase B) signaling pathways [14]. Stimulation of these pathways can lead to activation of unliganded ERα [15], with over-expression of EGFR and ErbB2 implicated in the failure of anti-estrogen therapy in both model systems [16-18] and clinical breast cancers [19-21]. ERRα1 can exist as a phosphoprotein [22]. SK-BR-3 cells, in which ERRα1 functions as a constitutive activator [12], contain 128-fold higher levels of ErbB2 mRNA than do MCF-7 cells [23], in which it functions as a down-modulator of transcription [13]. However, it is not known whether the ErbB2 signal transduction pathway or phosphorylation is involved in regulating ERRα activity.

Delineating the mechanism of ERRα regulation will provide new diagnostic and treatment tools for breast cancer.

### BRIEF SUMMARY OF THE INVENTION

The present invention is based on the inventors' discovery that blocking the ErbB2 signal transduction pathway inhibits ERRα activity by reducing its phosphorylation. In one aspect, the present invention relates to a method for determining whether a breast cancer patient is likely to respond to hormonal-blockade therapy or ErbB2-based therapy by analyzing the phosphorylation status of ERRα in breast cancer cells of the patient wherein the hyper-phosphorylation status indicates that the patient is likely to respond to ErbB2-based therapy but not to hormonal-blockade therapy.

In another aspect, the present invention relates to a method for treating breast cancer by reducing ERRα activity in patients with hyper-phosphorylated ERRα.

In still another aspect, the present relates to a method of determining prognosis of a breast cancer patient by analyzing phosphorylation status of ERRα in breast cancer cells of the patient wherein hyper-phosphorylation status indicates a poor clinical course and hypo-phosphorylation status indicates a more favorable clinical course.

In yet another aspect, the present invention relates to a method of identifying an agent that can modulate ERRα phosphorylation. The method involves exposing a group of cells that express ERRα to a test agent and comparing the phosphorylation status of ERRα in the cells to that of control cells that are not exposed to the agent wherein a difference in the phosphorylation status of ERRα indicates that the agent can modulate ERRα phosphorylation.

### BRIEF DESCRIPTION OF THE SEVERAL VIEWS OF THE DRAWINGS

FIG. 1 shows modulation of ERE-regulated transcription by ERRα1 via the EGFR/ErbB2 signaling pathways. Only a few of the numerous players in the EGFR/ErbB2 signaling pathways are indicated, along with the steps in these pathways blocked by the drugs used in Example 1 below.

FIG. 2 shows transcriptional activity of ERRα1 in low ErbB2-expressing MCF-7 cells versus high ErbB2-expressing BT-474 cells. (A) MCF-7 and (B) BT-474 cells were co-transfected in parallel with the ERE(5×)-regulated and TATA dual-luciferase reporter gene sets along with ERRα1, ERRα1_{L413A/L418A}, GRIP1 (glucocorticoid receptor interacting protein 1, SRC2) or empty parental expression plasmids as indicated. Transfected cells were incubated for 48 h in estrogen-free medium supplemented with ethanol, the solvent for the ligands, 100 pM E₂, or 100 pM E₂ plus 100 nM fulvestrant (ICI 182,780, faslodex) prior to being harvested for dual-luciferase assays. The results shown here are means±standard deviations from data obtained in one representative experiment performed in triplicate.

FIG. 3 shows phosphorylation of ERRα1 in vitro with (A) activated MAPK and Akt, and (B) that phosphorylation occurs in multiple regions of ERRα, including in ERRα's carboxy-terminal co-activator binding domain. (A) Equal amounts of ERRα or myelin basic protein (MBP) as a positive control were incubated in parallel with activated MAPK1, MAPK2, Akt1 and Akt2 as well as with [γ³²P]-ATP as described in Example 1 below. The products were resolved by sodium dodecylsulfate—(4-12%) polyacrylamide gel electrophoresis (SDS—(4-12%) PAGE) and visualized by autoradiography. (B) The indicated GST-ERRα1 fusion proteins (equimolar amounts) along with PHAS-I and GST-βglobin (MW=41 kDa), as positive and negative controls, respectively, were incubated with activated MAPK2 and [γ³²P]-ATP as described in Example 1 below. The products were resolved by SDS—(12%) PAGE and visualized with a PhosphorImager.

FIG. 4 shows autoradiograms of 2-dimensional (2D) gels showing the phosphorylated states of ERRα1 in MCF-7 cells, BT-474 cells, and two exposures (lighter and darker) of BT-474 cells incubated with MAb 4D5, a murine monoclonal antibody against ErbB2. MCF-7 and BT-474 cells were transfected in parallel with the wild-type ERRα1 expression plasmid. Twenty-seven hours later, the cells were metabolically labeled by incubation for 4 h in phosphate-free medium supplemented with [γ³²P]-orthophosphate. Afterward, whole-cell extracts were prepared. ERRα1 was immunoprecipitated with an anti-GST-hERRα1 polyclonal antiserum and resolved by 2D polyacrylamide gel electrophoresis. The MAb 4D5-treated cells incorporated less ³²P because they were growth inhibited as reflected by the lighter exposure; a second, darker exposure of this same gel shows ERRα's differential phosphorylation states.

FIG. 5 shows effects of EGFR, ErbB2 and their downstream signaling components, MEK and PI3K, on ERRα1-mediated constitutive activation of ERE-regulated transcription in BT-474 cells. BT-474 cells were co-transfected in parallel with the ERE(5x)-regulated and TATA dual-luciferase reporter gene sets along with the indicated expression plasmids. In panel (A), the cells were incubated for 48 h in estrogen-free medium supplemented with 20 μg/ml non-specific mouse IgG, 20 μg/ml trastuzumab (anti-ErbB2 antibody), or 1 μM gefitinib (ZD1839, Iressa; small molecule EGFR tyrosine kinase inhibitor) as indicated. In panel (B), the cells were incubated for 48 h in estrogen-free medium supplemented with dimethyl sulfoxide as the drug vehicle control, 20 μM U0126 (MEK inhibitor), or 20 μM LY294002 (PI3K inhibitor) as indicated. After incubation for 48 h, the cells were harvested for dual-luciferase assays. Data were analyzed as described in the legend to FIG. 2. (C) Immunoblot analysis of protein extracts (20 μg per sample) prepared from BT-474 cells treated as in panels A and B above for luciferase activity. The membrane was probed with antibodies that react specifically with ERRα. Reactive proteins were visualized by enhanced chemiluminescence and autoradiography. (D) Immunoblot analysis as in panel C above, only the membrane was probed with antibodies that react specifically with phosphorylated Akt (p-Akt), total Akt, phosphorylated MAPK (p-MAPK) or total MAPK.

FIG. 6 shows the growth effects of trastuzumab and gefitinib in BT-474 versus MCF-7 cells. MCF-7 and BT-474 cultured in the presence of A, trastuzumab and B, gefitinib. Using 12-well plates, MCF-7 and BT-474 cells were seeded at 15,000 and 30,000 cells per well. Cells were cultured for 7 days in the presence of the indicated test agents with media changes at day 0, day 2 and day 5. Following the 7 day treatment course, cells were assayed for DNA content per well. The results shown represent the mean ± SEM of three replicate wells.

### DETAILED DESCRIPTION OF THE INVENTION

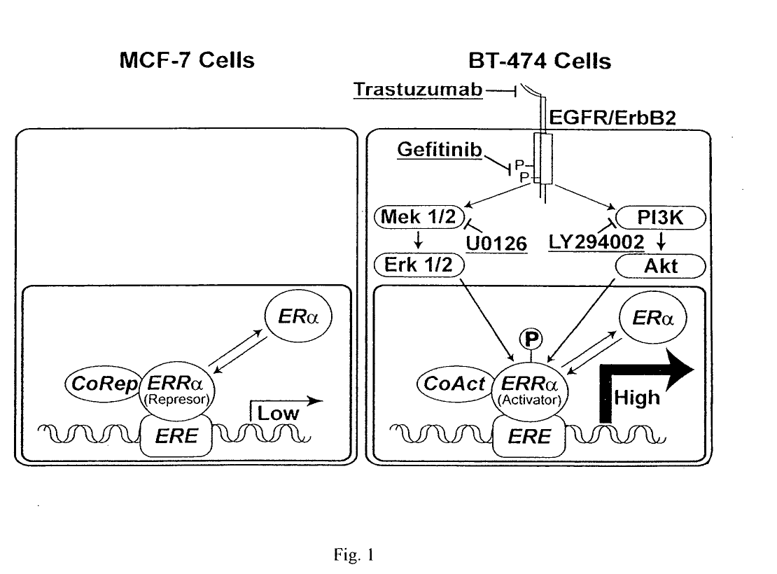

It is disclosed here that ERRα is a downstream target of ErbB2, whereby the ErbB2 signal transduction pathway activates ERRα by inducing its phosphorylation (FIG. 1). The resulting increased phosphorylated state of ERRα is referred to as hyper-phosphorylated ERRα (also called the activated form of ERRα), and a less phosphorylated state of ERRα is referred to as hypo-phosphorylated ERRα which may include unphosphorylated ERRα. The hyper-phosphorylated ERRα is an activator of estrogen response element (ERE)-mediated transcription and the hypo-phosphorylated ERRα is a repressor of ERE-mediated transcription. ERRα in its hyper-phosphorylated form in breast cancer cells indicates poor prognosis, responsiveness to ErbB2-based therapy and insensitivity to hormonal-blockade therapy.

As shown in Example 1 below, ERRα down-regulated ER-stimulated transcription in human mammary carcinoma MCF-7 cells, which are ERα -positive and do not overexpress ErbB2. In contrast, ERRα constitutively activated ERE-regulated transcription in the presence of the complete antiestrogen fulvestrant (ICI 182,780, faslodex) in human mammary carcinoma BT-474 cells, which are ERα-positive and overexpress ErbB2. Anti-ErbB2 antibodies inhibited the growth of BT-474 cells but not MCF-7 cells. ErbB2 activates its signal transduction through either homodimerization or through heterodimerization with another ErbB family member, such as the epidermal growth factor rector (EGFR). These ErbB2 complexes lead to phosphorylation of MEK (mitogen activated protein kinase kinase (MAPK kinase)), and in turn phosphorylation of MAPK; these factors are primary components in the MEK/MAPK pathway. In parallel, the ErbB2 complexes also lead to phosphorylation of phosphatidylinositol-3-OH kinase (PI3K), and in turn Akt; these factors are primary components in the PI3K/Akt pathway. Example 1 showed that ERRα was a substrate for phosphorylation by kinases such as MAPK. Example 1 further showed that disrupting the MEK/MAPK or PI3K/Akt signaling pathway with an anti-ErbB2 (also termed anti-HER2) monoclonal antibody (MAb), an EGFR kinase inhibitor, a MEK (MAPK kinase) inhibitor, or a PI3K inhibitor led to the inhibition of ERRα-stimulated ERE-regulated transcription in BT-474 cells. ERRα 's functional activity, that is to stimulate ERE-regulated transcription, was further shown to correlate with its extent of phosphorylation in BT-474 cells.

In one aspect, the present invention relates a method for determining whether a breast cancer patient is likely to respond to hormonal-blockade therapy or ErbB2-based therapy. The method involves analyzing the phosphorylation status of ERRα in breast cancer cells of the patient. Hyper-phosphorylation status of ERRα indicates that the patient is likely to respond to ErbB2-based therapy, but not to hormonal-blockade therapy. Such a patient should consider choosing ErbB2-based therapy over hormonal-blockade therapy. Hypo-phosphorylation status of ERRα indicates that the patient is less likely to respond to ErbB2-based therapy in comparison to a patient with hyper-phosphrylated ERRα, but likely to respond to hormonal-blockade therapy if ERα is present at a high level in the same breast cancer cells. Therefore, for a patient with ERα-positive and hypo-phosphorylated ERRα status, hormonal-blockade therapy should be favored over ErbB2-based therapy.

In one embodiment, the above method further involves measuring the ErbB2 expression level in the breast cancer cells of the patient. A high level of ErbB2 expression (ErbB2-positive) and hyper-phosphorylation status of ERRα indicate that the patient is likely to respond to ErbB2-based therapy. Such a patient should consider choosing ErbB2-based therapy.

In another embodiment, the above method further involves measuring the ERα expression level in the breast cancer cells of the patient. A high level of ERα expression (ER-positive) and hypo-phosphorylation status of ERRα indicate that the patient is likely sensitive to hormonal-blockade therapy. Such a patient should consider choosing hormonal-blockade therapy. On the other hand, a high level of ERα expression (ER-positive) and hyper-phosphorylation status of ERRα indicate that the patient is likely insensitive to hormonal-blockade therapy by itself. Such a patient should choose ErbB2-based therapy in combination with hormonal-blockade therapy.

In another aspect, the present invention relates to a method of treating breast cancer. The method involves analyzing the phosphorylation status of ERRα in the breast cancer cells of a patient and when hyper-phosphorylated ERRα is found, reducing the ERRα activity, such as its ability to stimulate ERE-mediated transcription, in the patient. ERRα activity in the patient can be reduced by lowering the level of ERRα, blocking phosphorylation of ERRα, or treating the patient with an antagonist ligand of ERRα such as XCT790 [24] (reference 24 is herein incorporated by reference in its entirety). ERRα phosphorylation can be reduced by blocking the ErbB2 signaling pathway. The ErbB2 signaling pathway or its downstream signaling intermediates can be blocked, for example, by blocking ErbB2 activity, EGFR activity, MEK (MAPK kinase) activity, MAPK activity, PI3K activity, Akt activity, or a combination of any of the foregoing.

In another aspect, the present relates to a method of determining prognosis of a breast cancer patient. The method involves analyzing the phosphorylation status of ERRα in breast cancer cells of the patient. Hyper-phosphorylation status of ERRα indicates a poor clinical course and hypo-phosphorylation status of ERRα indicates a more favorable clinical course. This means that after diagnosis, breast cancer patients with hypo-phosphorylated ERRα are more likely to respond to hormonal-blockade therapy and have a longer overall survival time than those with hyper-phosphorylated ERRα.

In another aspect, the present invention relates to a method of identifying an agent that can modulate the phosphorylation of ERRα. The method involves exposing a group of cells that express ERRα to a test agent and comparing the phosphorylation status of ERRα in the cells to that of control cells that are not exposed to the agent wherein a difference in the phosphorylation status of ERRα indicates that the agent can modulate the phosphorylation of ERRα.

There are many ways that a skilled artisan can analyze the phosphorylation status of ERRα. All of them can be used in the present invention. Methods of using 2-D gels and antibodies to differentiate hyper- and hypo-phosphorylated ERRα are described in examples 1 and 2 below, respectively. With the 2-D gel method, a primary culture of breast cancer cells and optionally a primary culture of surrounding normal breast epithelial cells from a patient can be established and labeled as described in example 1. The 2-D gel for breast cancer cells can be compared to that of normal breast epithelial cells and the disappearance or a reduction in the level of one or more hypo-phosphorylated isoforms of ERRα and/or an increase in the level of one or more hyper-phosphorylated isoforms of ERRα indicates hyper-phosphorylation status. Besides normal breast epithelial cells from the same patient, other standards or controls can also be used. For example, normal breast epithelial cells and/or breast cancer cells from other patients can be used to establish ERRα phosphorylation standards. Alternatively, bacterially expressed recombinant ERRα could be used as a non-phosphorylated standard. Also, a phosphorylated ERRα standard could be prepared in vitro by incubating bacterially expressed recombinant ERRα with activated MAPK and Akt as described in Example 1. It is understood that different isoforms of ERRα (in terms of phosphorylation status) on a 2-D gel can also be analyzed by Western blot with an ERRα antibody that can recognize all isoforms of ERRα. In this case, primary culture and the radioisotope labeling thereof are not necessary. Once phosphospecific anti-ERRα antibodies are available, they could be used to probe blots of 1-D gels, or even dot blots for phosphorylation status. They could also be used in immunohistochemical analysis of paraffin-embedded tissue sections of tumors.

There are many ways that a skilled artisan can determine the expression level of ErbB2, ERRα, ERα and PgR. All of them can be used in the present invention. ERRα may be expressed as one of the two isoforms, ERRα1 and ERRα2, and possibly other, as-yet-unidentified isoforms of ERRα. Unless specifically mentioned, the expression level of ERRα in the specification and claims refers to the cumulative expression level of all ERRα isoforms. For example, to assess the ERRα expression level, polymerase chain reaction (PCR) primers that can amplify both ERRα1 and ERRα2 are used. Antibodies that recognize both ERRα1 and ERRα2 can also be used to measure the ERRα expression level. When the expression level of ErbB2, ERRα and ERα are referred to as high or low in the specification and claims, it is measured against a reference level, such as the median level of ErbB2, ERRα and ERα obtained from breast cancer tissues of a group of patients. The more breast cancer tissue samples of different breast cancer patients are used to establish the median level, the more accurate the median level is. Preferably, the median level is obtained from analyzing breast cancer tissues of at least 25 patients assuming an appropriate level of statistical significance can be achieved. Specifically for ErbB2, a high level or positive status can also be a status that is associated with poor prognosis such as determined by the various methods described in Dowsett, M., T. Cooke, et al. (2000) “Assessment of HER2 status in breast cancer: why, when and how?” Eur. J. Cancer 36(2): 170-6, and in Pauletti, G., S. Dandekar, et al. (2000) “Assessment of methods for tissue-based detection of the HER-2/neu alteration in human breast cancer: a direct comparison of fluorescence in situ hybridization and immunohistochemistry” J. Clin. Oncol. 18(21): 3651-64, both of which are herein incorporated by reference in their entirety. For example, as described in Dowsett, M., T. Cooke, et al., ErbB2 gene amplification can be considered a high level or positive status for the purpose of the present invention.

ErbB2-based therapy includes the use of agents that either directly inhibit ErbB2 activity (anti-ErbB2 antibodies such as trastuzumab (Herceptin) and ErbB2-specific kinase inhibitors) or indirectly inhibit ErbB2 activity by inhibiting the activity of other ErbB members that heterodimerize with ErbB2. For instance, ErbB2 and EGFR can form ErbB2-EGFR heterodimers to transduce signals. Anti-EGFR antibodies and EGFR kinase inhibitors such as gefitinib (ZD1839, Iressa) can thus be used to indirectly block ErbB2 activity.

The MEK/MAPK signaling pathway downstream of ErbB2 can be disrupted by inhibiting the activity of ErbB2 and/or the activity of one or more downstream components of the pathway such as MEK (MAPK kinase), MAPK and ERRα.

The PI3K/Akt signaling pathway downstream of ErbB2 can be disrupted by inhibiting the activity of ErbB2 and/or the activity of one or more downstream components of the pathway such as PI3K, Akt and ERRα.

Hormonal blockade therapy includes the use of antiestrogens or selective estrogen receptor modulators (SERMs), aromatase inhibitors and other agents that can block the production or activity of estrogens.

The present invention will be more readily understood upon consideration of the following examples which are exemplary and are not intended to limit the scope of the invention.

### EXAMPLE 1

### ERRα as a Biomarker and Treatment Target for Breast Cancer

This example demonstrates that signaling via EGFR and ErbB2 leads to phosphorylation of ERRα, thereby modulating its activities (FIG. 1). In particular, the example shows that ERRα1 constitutively activates ERE-regulated transcription in ER-positive, high ErbB2-expressing BT-474 mammary carcinoma cells [23], even in the presence of the complete anti-estrogen fulvestrant (ICI 182,780, faslodex). Mitogen-activated protein kinases (MAPKs) and Akts phosphorylated ERRα1 in vitro. Disruption of either ErbB2 signaling using trastuzumab (Herceptin) [25], an anti-ErbB2 monoclonal antibody, or disruption of epidermal growth factor receptor (EGFR) signaling using gefitinib (ZD1839, Iressa) [26], a small molecule tyrosine kinase inhibitor, reduced ERRα1's ability to activate transcription in BT-474 cells. The MEK (MAPK kinase) inhibitor U0126 [27] and the phosphatidylinositol-3-OH kinase (PI3K) inhibitor LY294002 [28], inhibitors of downstream components in ErbB2 and EGFR signaling, respectively, also reduced ERRα1's ability to activate transcription. Furthermore, the difference in ERRα1's functional activity positively correlated with the extent of its phosphorylation in situ. Thus, ERRα1's activity is regulated at least in part via ErbB2 and EGFR signaling. Because over-expression of either ErbB2 or EGFR in human breast cancer indicates aggressive disease and associates with resistance to hormonal-blockage therapies, ERRα1's phosphorylation status can indicate sensitivity of tumors to hormonal and ErbB2/EGFR-based therapeutic drugs such as tamoxifen and trastuzumab. Hence, ERRα can be used as a biomarker of clinical outcome, predictor of therapeutic benefit, and target for new anti-cancer agents.

Materials and Methods

Plasmids: Plasmid pcDNA3.1-hERRα1, encoding full-length, 423-amino acids human ERRα1, the major isoform of ERRα, and plasmid pcDNA3.1-hEPRRα1_{L413A/L418A}, encoding a variant of ERRα1 defective in the C-terminal coactivator-binding LXXLL motif, have been previously described [13]. Plasmid pcDNA3-GRIP1 encodes the coactivator GRIP1 [29].

Plasmids pERE(5x)-ffLuc and pTATA-ffLuc, encoding firefly luciferase, and plasmid pTATA-srLuc, encoding renilla luciferase, were used as matched dual-luciferase reporter sets to assay ERE-regulated versus basal transcription along with an internal control. They were constructed by insertion via HindIII linkers of the nts −31 to +31 region of the herpes simplex virus thymidine kinase (HSV-TK) promoter [30] into pGL3-Basic and phRG-B (Promega, Madison, Wis.), respectively. Plasmid pERE(5x)-ffLuc, containing five tandem copies of the consensus palindromic ERE, was then constructed by insertion into pTATA-fELuc of the oligodeoxynucleotides 5′-tcgagAGAGGTCACTGTGACCTCTgagAGAGGTCACTGTGACCTCTctcAGAGGTCACTGT GACCTCTgcgAGAGGTCACTGTGACCTCTgcgAGAGGTCACTGTGACCTCTa-3′ (SEQ ID NO:1) and 5′-gatctAGAGGTCACAGTGACCTCTcgcAGAGGTCACAGTGACCTCTcgcAGAGGTCACAGT GACCTCTgagAGAGGTCACAGTGACCTCTctcAGAGGTCACAGTGACCTCTc-3′ (SEQ ID NO:2). EREs are indicated by upper case letters, with core half-sites underlined.

Cells: The MCF-7 cells used here are MCF-7/WS8, a clonal derivative that maintains estrogen responsiveness [31]. BT-474 cells were obtained from the American Type Culture Collection (Manassas, Va.). Except where indicated otherwise, MCF-7 and BT-474 cells were cultured in phenol red-containing RPMI-1640, 10% whole fetal bovine serum (FBS), 6 ng/ml insulin, 2 mM glutamine, 100 μM non-essential amino acids, and 100 U of penicillin and streptomycin per ml. Estrogen-free medium contained charcoal-stripped FBS [32] and phenol red-free RPMI-1640 in place of whole FBS and standard RPMI-1640. Cells were maintained at 37° C. in a humidified 5% CO₂ atmosphere.

Transient transfection and dual-luciferase assays: MCF-7 and BT-474 cells were maintained under estrogen-free conditions for 4 days before being seeded in 24-well plates at densities of 100,000 and 150,000 cells/well, respectively. The next day the cells were placed in fresh medium containing the indicated test agents immediately before transfection. 17β-estradiol (Sigma-Aldrich, St. Louis, Mo.) and the complete anti-estrogen fulvestrant (ICI 182,780, faslodex; a generous gift from AstraZeneca, Macclesfield, UK) were dissolved in ethanol. Control, non-specific murine IgG (reagent grade, Sigma-Aldrich) was dissolved in phosphate-buffered saline. Trastuzumab (Herceptin), dissolved in bacteriostatic water (pH 4.5-7.0), was purchased from the Robert H. Lurie Comprehensive Cancer Center Pharmacy (Chicago, Ill.). Gefitinib (a generous gift from AstraZeneca) was initially dissolved in DMSO, followed by dilution in ethanol. U0126 (Promega, Madison, Wis.) and LY294002 (Promega) were dissolved in DMSO. All test agents were added to the medium at a 1:1000 (v/v) dilution.

Cells were co-transfected using TransIT LT1 reagent (Mirus, Madison, Wis.) along with the following DNAs as indicated: 150 ng of pcDNA3.1-hERRα1, pcDNA3.1-hERRα1_{L413A/L418A} or their empty parental plasmid, pcDNA3.1; 200 ng of pGRIP1 or its empty parental plasmid, pcDNA3.1; 200 ng of pERE(5x)-ffLuc or pTATA-ffLuc; and 50 ng of pTATA-srLuc. Data are presented as luciferase activity from cells co-transfected with the ERE-regulated reporter set (pERE(5x)-ffLuc internally normalized to pTATA-srLuc), with external normalization to the luciferase activity from cells co-transfected in parallel with the basal promoter reporter set (pTATA-ffLuc internally normalized to pTATA-srLuc). Each co-transfection was performed in triplicate, with each set of experiments being performed on three separate occasions. Numbers shown are means ± standard deviations of data obtained from single representative experiments.

In vivo [³²P]-labeling and 2D PAGE: MCF-7 and BT-474 cells at about 70% of confluency were transiently transfected with pcDNA3.1-hERRα1 (3 μg per 10-cm dish). The medium in one dish of BT-474 cells was supplemented with MAb 4D5 (2.5 μg/ml). A hybridoma cell line that secretes MAb 4D5, a murine precursor of Herceptin directed against the ectodomain of ErbB2 (HER2), was obtained from the American Type Culture Collection. MAb 4D5 was isolated from the ascites fluid of a mouse inoculated with this cell line via IgG purification. Twenty-four hours after transfection, the cells were washed twice with phosphate-free RPMI-1640 (Specialty Media, Phillipsburg, N.J.), incubated in phosphate-free RPMI-1640 for 3 h at 37° C., and metabolically labeled by addition of 2.5 mCi/dish of ³²P-labeled orthophosphoric acid (9,000 Ci/mmol, NEN Life Science) and incubation for an additional 4 h. Afterward, the cells were washed twice with phosphate-free RPMI-1640 and lysed by incubation for 20 min at 4° C. in 600 μl lysis buffer (50 mM Tris-HCl (pH 7.5), 150 mM NaCl, 1 mM EDTA, 1% NP40, 0.1% SDS, 0.5 mM phenylmethylsufonyl fluoride (PMSF)). The lysate was passed through a 27-gauge needle and centrifuged for 5 min at 4° C. The resulting supernatant was incubated with 45 μl of protein-A agarose (Santa Cruz Biotech, Santa Cruz, Calif.) for 1 h at 4° C. and centrifuged again. ERRα1 was immunoprecipitated from this second supernatant by incubation for 1 h with 1 μl of polyclonal antiserum raised against GST-ERRα₁₁₇₋₃₂₉ [33], followed by incubation for 1 h with 45 μl protein A-conjugated agarose beads and centrifugation. The immunoprecipitate was washed twice with 20 mM Tris-HCl (pH 8.0), 200 mM NaCl, 1 mM EDTA, 0.25% NP40 and 0.5 mM PMSF. ERRα1 was eluted by incubation at 100° C. in 12 μl of 2×SDS loading buffer and resolved by 2-dimensional (2D) gel electrophoresis (Kendrick Laboratory, Inc., Madison, Wis.) performed with pH 3.5-10 isoelectric focusing and SDS-12% polyacrylamide gels.

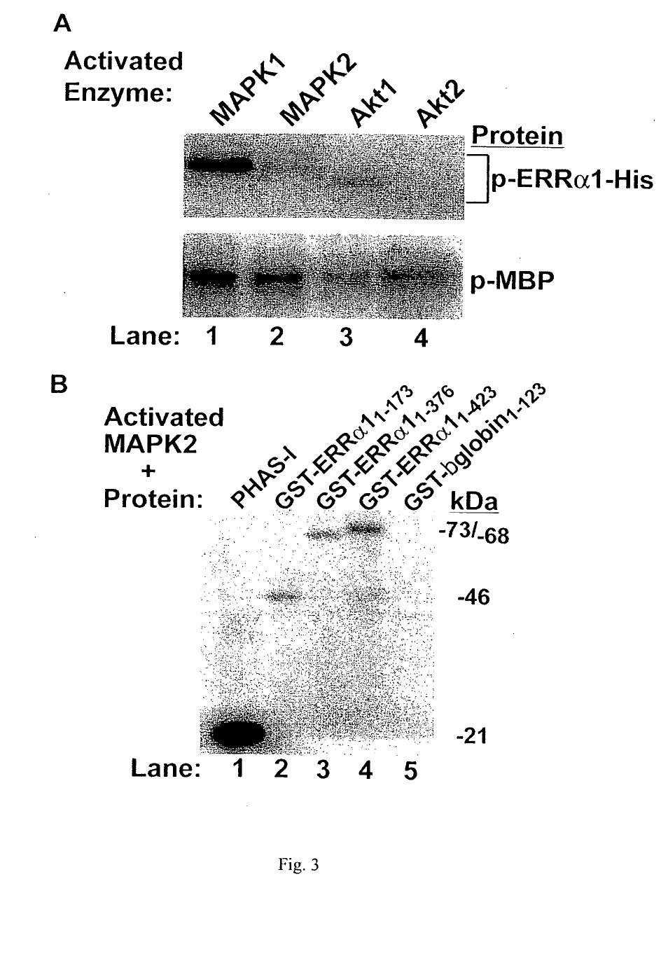

Protein kinase assays: In FIG. 3A, activated forms of MAPK1, MAPK2, Akt1, and Akt2 were each incubated in parallel with ERRα1 and [γ-³²P]ATP. Human MAPK1 and human MAPK2, both containing an N-terminal GST tag, were expressed and purified from E. coli, followed by activation with MEKI (Upstate Cell Signaling Solutions, Lake Placid, N.Y.). Human Akt1 and human Akt2, each containing an N-terminal 6×His tag and lacking amino acids 1-117 (Plecktrin Homology domain), were expressed and purified from Sf21 cells (Upstate Cell Signaling Solutions). In addition, these kinases were activated by means of mutations; serine 473 had been mutated to aspartic acid in Akt1, and serine 474 had been mutated to aspartic acid in Akt2. Human ERRα1, containing a C-terminal 6×His tag, was expressed and purified from E. coli using Ni—NTA agarose (Qiagen, Valecia, Calif.). Myelin basic protein (MBP) was obtained from Upstate Cell Signaling Solutions. In the MAPK phosphorylation assays, the reactions occurred in a buffer containing 20 mM MOPS, pH 7.2, 25 mM β-glycerol phosphate, 5 mM EGTA, 1 mM sodium orthovanadate, 1 mM dithiothreitol, 27 nM MgCl₂, and 180 μM ATP. In the Akt phosphorylation assays, the reactions occurred in a buffer containing 50 mM Tris-HCl, pH 7.5, 0.1 mM EGTA, 15 mM dithiotreitol, 27 nM MgCl₂, and 180 μM ATP. In both the MAPK and Akt phosphorylation reactions, 0.05 units of each kinase was incubated with 1.5 μg ERRα1 and 1 μCi of [γ-³²P]ATP (3 mCi/μmol) at 30° C. for 30 min in a total volume of 25 μl. In the positive control reactions containing MPB, 20 μg of the MBP was used in place of the ERRα1 protein. The products were resolved by SDS-(4-12%) PAGE. In FIG. 3B, rat p42 MAPK2 (ERK2; Calbiochem, San Diego, Calif.) that had been phosphorylated in vitro by a constitutively active MEK1 mutant was used as the source of activated MAPK. Full-length and deleted variant GST-ERRα1 and GST-βglobin₁₋₁₂₃ fusion proteins were expressed and purified from E. coli as previously described [34]. MAPK2 phosphorylation assays were performed at 30° C. for 30 min in 40 μl reactions containing 12 units (20 ng) of activated ERK2, 1 μCi of [γ-³²P]ATP (5 mCi/μmol), and 1.0 μg of GST-ERRα1₁₋₄₂₃, 1.08 μg of GST-ERRα1_{1-376,} 1.6 μg of GST-ERRα1₁₋₁₇₃, 1 μg of PHAS-1 (Calbiochem), or 1 μg of GST-βglobin₁₋₁₂₃ in 25 mM HEPES (pH 7.5), 10 mM MgOAc, 50 μM ATP. The products were resolved by SDS-12% PAGE.

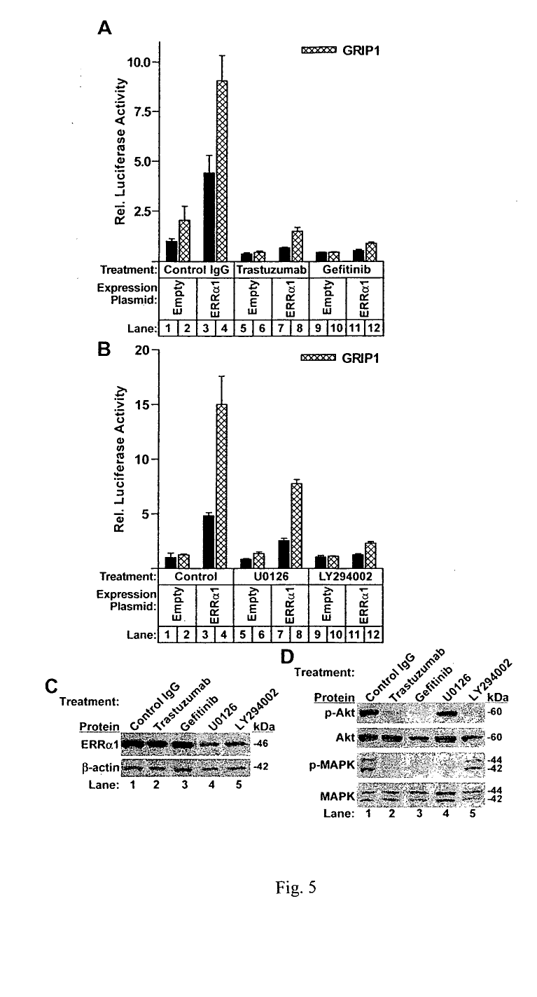

Immunoblot analyses: BT-474 cells, maintained for 5 days in estrogen-free medium and grown to about 70% of confluency, were transiently transfected with pcDNA3.1-hERRα1 (5 μg per 10-cm dish), and then incubated for 48 h in estrogen-free medium supplemented with 20 μg/ml mouse IgG, 20 μg/ml trastuzumab, 1 μM gefitinib, 20 μM U0126, or 20 μM LY294002. Proteins were extracted in Cell Lysis Buffer (Cell Signaling Technology, Beverly, Mass.) supplemented with Protease Inhibitor Cocktail Set III and Phosphatase Inhibitor Cocktail Set II (Calbiochem). Protein amounts were quantified with Coomassie Protein Assay Reagent (Pierce Biotechnology) with BSA as the standard. Protein (20 μg per extract) was resolved by SDS-12% PAGE and electroblotted onto a nylon membrane. In FIG. 5C, the membrane was probed sequentially with the anti-GST-ERRα₁₁₇₋₃₂₉ poluclonal antibody [33] and β-actin polyclonal antibody (Sigma). In FIG. 5D, the membrane was probed sequentially with p44/42 MAPK polyclonal antibody, phospho-specific p44/42 MAPK (Thr202/Tyr204) monoclonal antibody E10, Akt polyclonal antibody, and phospho-specific Akt (Ser473) polyclonal antibody (Cell Signaling Technology). Reacting material was detected using horseradish peroxidase-conjugated secondary antibodies (Jackson ImmunoResearch Laboratories, West Grove, Pa.) together with enhanced chemiluminescence (Amersham Pharmacia) and autoradiography.

Results

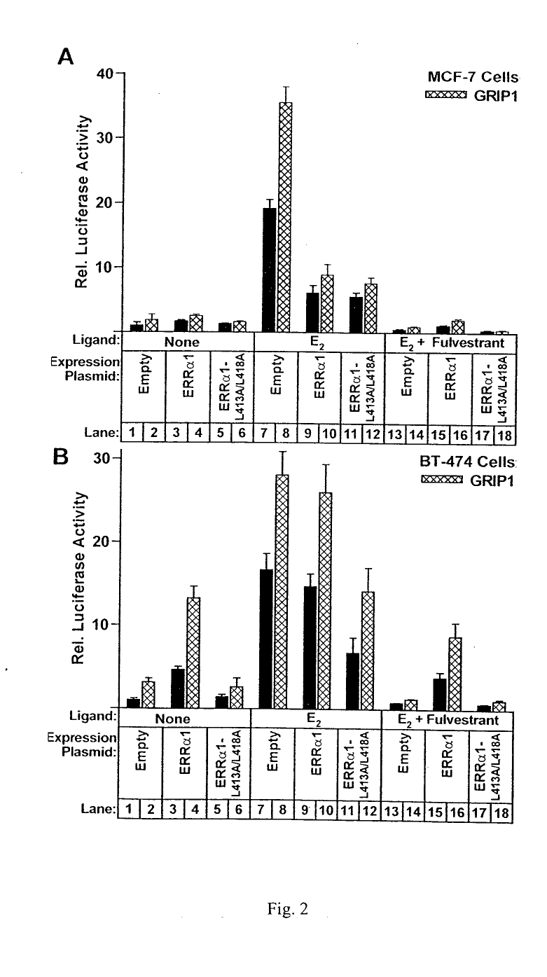

ERRα1 constitutively activates transcription in BT-474 cells, functionally substitutingfor ER. To examine ERRα1's abilities to modulate ERE-regulated transcription, we co-transfected low ErbB2-expressing MCF-7 cells and high ErbB2-expressing BT-474 cells with the reporter and expression plasmids indicated in FIG. 2 and, subsequently, cultured them for 48 h in estrogen-free medium supplemented with the indicated ligands. As expected, incubation of ER-positive MCF-7 cells with 100 pM E₂ induced ERE-regulated transcription 19 fold (FIG. 2A, lane 7 vs. 1). Introduction of exogenous wild-type ERRα1 or ERRα1_{L413A/L418A}, a variant of ERRα1 defective in the C-terminal coactivator docking site [8, 12], led to a 68% and 71% reduction, respectively, in ER-stimulated transcription in these cells (FIG. 2A, lanes 9 and 11 vs. 7). Over-expression of the coactivator GRIP1 stimulated ER-mediated transcription an additional 1.8-fold (FIG. 2A, lane 8 vs. 7). However, down-modulation of ER-stimulated transcription by ERRα1 was even greater, i.e., 75% to 78%, when GRIPI was over-expressed (FIG. 2A, lanes 10 and 12 vs. 8). Thus, confirming prior findings [13], ERRα1 efficiently down-modulates ER-mediated transcription in MCF-7 cells.

ERRα1's activities were also examined in the absence of ER-stimulated transcription by incubation of the MCF-7 cells in the absence of E₂ (FIG. 2A, lanes 1-6), and the presence of 100 pM E₂ along with 100 nM of the complete anti-estrogen fulvestrant (FIG. 2A, lanes 13-18). Under either of these conditions, over-expression of wild-type ERRα1 led to a barely significant 1.3- to 1.7-fold increase in transcription (FIG. 2A, lanes 3, 4, 15, and 16 vs. lanes 1, 2, 13, and 14). This small activation was absent in the co-activator-binding mutant (FIG. 2A, lanes 5, 6, 17, and 18 vs. lanes 1, 2, 13, and 14). Thus, in the absence of functional ER, ERRα1 exhibits weak transcriptional activator activity in MCF-7 cells, likely via its carboxy-terminal coactivator-binding motif [7], in addition to strong, active repression activity via its repressor domain(s) [13].

We examined likewise ERRα1's ability to modulate ERE-regulated transcription in BT-474 cells (FIG. 2B). In contrast to the findings obtained in MCF-7 cells, over-expression of wild-type ERRα1 in BT-474 cells failed to lead to down-modulation of ER-stimulated transcription whether or not GRIP1 was also over-expressed (FIG. 2B, lanes 9 and 10 vs. lanes 7 and 8). Rather, in the absence of ER-stimulated transcription, over-expression of wild-type ERRα1 led to an approximately 5-fold increase or 11- to 13-fold increase in ERE-regulated transcription in the absence and presence of over-expressed GRIP1, respectively (FIG. 2B, lanes 3, 4, 15, and 16 vs. lanes 1, 2, 13, and 14), an increase that did not occur with ERRα1_{L413A/L418A} (FIG. 2B, lanes 5, 6, 17, and 18 vs. lanes 1, 2, 13, and 14). Interestingly, introduction of ERRα1_{L413A/L418A} led to a 50% to 60% reduction in E₂-stimulated transcription (FIG. 2B, lanes 11 and 12 vs. lanes 7 and 8). Thus, ERRα1 constitutively activates ERE-regulated transcription in BT-474 cells to a level similar to that observed with ERα, with this activity likely occurring in part via its carboxy-terminal coactivator-binding motif. We conclude that the apparent lack of effect of wild-type ERRα1 on ER-stimulated transcription in the high ErbB2-expressing BT-474 cells is likely due to it simply substituting for ERα in activating transcription when it out-competes ERα for binding to the EREs on the reporter plasmid. On the other hand, ERRα1 down-modulates ER-stimulated transcription in MCF-7 cells because it provides repressor or lower overall activator activity than ERα when it out-competes ERα for binding to the EREs in these low ErbB2-expressing cells.

Activated MAPK and Akt phophorylate ERRα1 in vitro. How does ERRα1 function as a repressor of ERE-regulated transcription in some cell types, yet as an activator in other cell types? Sladek et al. [22] reported that ERRα1 can exist as a phosphoprotein. To begin to identify signaling pathways that may affect ERRα1's state of phosphorylation, we tested whether ERRα1 can serve as an in vitro substrate of MAPK and Akt. Equal amounts of E. coli-produced c-terminal histidine-tagged ERRα (ERRα-His) were incubated in parallel with activated MAPK1, MAPK2, Akt1 and Akt2 in the presence of [γ-³²P]-ATP followed by resolution of phosphorylated products by SDS—(4-12%) PAGE. Each of the kinases tested was capable of phosphorylating ERRα in vitro. Although this assay was not quantitative, it suggests that ERRα may exhibit specificity as a kinase substrate as judged by its apparent differential molecular weight. ERRα may be phosphorylated at an increasing number of sites by Akt1 versus Akt2 and again by MAPK1 and MAPK2 versus Akt1 as suggested by ERRα's apparent increase in molecular weight. However, it should be noted that the extent of phosphorylation of ERRα observed in vitro with individual kinases assayed one at a time (FIG. 3A) does not equal the maximum extent observed in living cells in which ERRα is likely phosphorylated concurrently by more than one kinase (compare with FIG. 5C). To begin to localize sites of phosphorylation by MAPK1, full-length (GST-ERRα1₁₋₄₂₃) and truncated variants (GST-ERRα1_{1-376,} GST-ERRα1₁₋₁₇₃) of GST-ERRα1 fusion proteins were synthesized in and purified from E. coli. PHAS-1 (phosphorylated heat and acid-stable protein regulated by insulin) and GST-β-globin₁₋₁₂₃ served as positive and negative controls, respectively. Equimolar amounts of each protein were incubated in parallel with activated p42 MAPK (ERK2) and [γ32P]-ATP and resolved by 12% SDS-PAGE (FIG. 3B). As expected, activated MAPK efficiently phosphorylated PHAS-1, but not GST-β-globin₁₋₁₂₃. Interestingly, MAPK phosphorylated each of the GST-ERRα1 fusion proteins, with more label being incorporated into GST-ERRα1₁₋₄₂₃ than into GST-ERRα1₁₋₃₇₆ and GST-ERRα1₁₋₁₇₃. Thus, ERRα1 can serve as a substrate of MAPK1/2 and Akt1/2, with multiple phosphorylation sites likely present within the protein, including at least one within the carboxy-terminal activation domain.

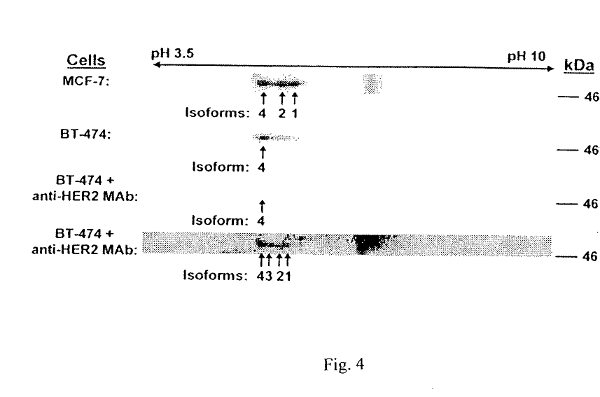

Differential phosphorylation of ERRα in BT-474 vs. MCF-7 cells. To begin to test the hypothesis that ERRα1's activities may be regulated in part by post-translational phosphorylations, we examined ERRα1's phosphorylation status by incubation of MCF-7 and BT-474 cells with ³²P-orthophosphate, immunoprecipitation of the metabolically labeled ERRα1 with an ERRα-specific serum, and separation of its isoforms by 2D-PAGE. While MCF-7 cells were found to contain roughly similar amounts of three ³²P-labeled isoforms of ERRα1, most of the ³²P phosphate-labeled ERRα1 in BT-474 cells was present in the most acidic of these three isoforms (FIG. 4). Hence, BT-474 cells predominantly contain one multiply-phosphorylated isoform of ERRα1, while MCF-7 cells contain several, differently phosphorylated isoforms of ERRα1, including, possibly, an un-phosphorylated isoform that would not have been detected in this experiment. Furthermore, treatment of BT-474 cells with a monoclonal antibody that disrupts the ErbB2 signaling pathway led to the appearance of several, differently phosphorylated isoforms of ERRα1, somewhat similar to the pattern observed with MCF-7 cells. As shown below (FIG. 5A), this type of treatment also converts ERRα1 from an activator to a repressor of ERE-regulated transcription. Thus, we conclude that the hypo-phosphorylated isoforms of ERRα1 correlate with repressor functionality while the hyper-phosphorylated isoform correlates with activator functionality.

Inhibition of EGFR and ErbB2 signaling abrogates ERRα1's activation function. Given that the phosphorylated state of ERRα1 can be altered via the ErbB2/MAPK signaling pathway, we next examined whether ERRα1's transcriptional activities are also affected by this pathway. Incubation with either the EGFR inhibitor gefitinib (ZD1839, Iressa) or the ErbB2 inhibitor trastuzumab (Herceptin) is known to significantly inhibit the growth of BT-474 cells [26], but to affect only minimally the growth of MCF-7 cells [35]. To test directly whether EGFR and ErbB2 signaling affects the activities of ERRα1, BT-474 cells were co-transfected with reporter and expression plasmids as described above and incubated for 48 h in estrogen-free medium supplemented with 1 μM gefitinib, 20 μg/ml trastuzumab, or 20 μg/ml non-specific murine IgG as a control (FIG. 5A). In the control-treated cells, over-expression of ERRα1 led to a 4- to 5-fold activation of ERE-regulated transcription as expected (FIG. 5A, lanes 3 and 4 vs. 1 and 2). Incubation with trastuzumab led to an approximately 85% inhibition of ERRα1's activity (FIG. 5A, lanes 7 and 8 vs. lanes 3 and 4) to a level below that observed in the presence of only endogenous ERRα1 (FIG. 5A, lanes 7 and 8 vs. lanes 1 and 2), likely due to trastuzumab treatment affecting the activity of endogenous ERRα1 as well (FIG. 5A, lanes 5 and 6 vs. 1 and 2). Incubation with gefitinib led to an even greater inhibition, i.e., approximately 90%, of ERRα1's activator activity (FIG. 5A, lanes 11 and 12 vs. lanes 3 and 4). Over-expression of GRIP1 largely failed to reverse this inhibition by drug treatment (FIG. 5A, even-numbered lanes). Immunoblot analysis of BT-474 cells transfected with ERRα showed that its expression was maintained in the presence of trastuzumab and gefitinib (FIG. 5C, lanes 1-3). Also, immunoblot analysis with antisera specific to the phosphorylated versus unphosphorylated forms of MAPK and Akt confirmed that these drug treatments had, indeed, inhibited activation of the MEK/MAPK and PI3K/Akt signaling pathways in these cells (FIG. 5D, lanes 1-3). Because incubation of BT-474 cells with a monoclonal antibody to ErbB2 also led to a significant decrease in the proportion of ERRα1 existing in its most hyper-phosphorylated isoform (FIG. 3), we conclude that disruption of these signaling pathways with either trastuzumab or gefitinib likely prevents ERRα1 from functioning as an activator of ERE-regulated transcription in BT-474 cells by inhibiting the addition of post-translational phosphorylations involved in ERRα1 functioning as an activator.

To test whether inhibition of EGFR and ErbB2 signaling modulates ERRα1's activity by affecting the activities of downstream components in these pathways, we examined the effects on ERRα1's activity of incubation of BT-474 cells with U0126 and LY294002, direct inhibitors of MEK and PI3K, respectively (see FIG. 1). In the control cells treated with only DMSO, the solvent for these drugs, over-expression of ERRα1 led, as above, to an approximately 5-fold activation of ERE-regulated transcription (FIG. 5B, lane 3 vs. 1). Incubation with U0126 led to an approximately 50% reduction in ERRα1's activator activity whether or not GRIP1 was also over-expressed (FIG. 5B, lanes 7 and 8 vs. lanes 3 and 4). The effect of incubation with LY294002 was even greater, inhibiting ERRα1-mediated activation of ERE-regulated transcription by 75% to 85% (FIG. 5B, lanes 11 and 12 vs. lanes 3 and 4). As a control, immunoblot analysis of ERRα-transfected BT-474 cells showed that ERRα was still expressed when the cells were treated with U0126 and LY294002 (FIG. 5C, lanes 4-5). Also, immunoblot analysis confirmed that treatment of these cells with U0126 blocked MEK, leading to inhibition of phosphorylation of MAPK without affecting Akt status (FIG. 5D, lane 4), while treatment with LY294002 blocked PI3K, leading to inhibition of phosphorylation of Akt without affecting MAPK status (FIG. 5D, lane 5). Therefore, the MEK/MAPK and PI3K/Akt signaling pathways likely contribute to ERRα1's ability to activate ERE-regulated transcription in BT-474 cells.

Combining the observations reported here and elsewhere [4, 12], we propose the following model for regulation of the transcriptional activities of ERRα1 (FIG. 1). In cells expressing ErbB2 at low levels (e.g., MCF-7), ERRα1 exists, on average, in a hypo-phosphorylated form, inhibiting transcription via interactions of its repressor domain(s) with cellular co-repressors when bound to response elements in DNA. In cells expressing ErbB2 at high levels (e.g., BT-474), ErbB2, likely as a heterodimer with EGFR, signals hyper-phosphorylation of ERRα1, at least in part through the MEK/MAPK and PI3K/Akt signaling pathways. This hyper-phosphorylated form of ERRα1 interacts with cellular coactivator(s) (e.g., GRIP1) via its activator domain(s), one of which is located within the carboxy-terminal end of the protein (FIGS. 2 and 3). Thus, phosphorylation of ERRα1 via the EGRF/ErbB2 signaling pathways converts it to a constitutive transcriptional activator capable of stimulating ERE-regulated transcription regardless of the presence of ERox, estrogens, or anti-estrogens including fulvestrant.

Over-expression of ErbB2 can confer resistance of mammary carcinoma cells to anti-estrogens even when functional ER is present, while treatment with MAPK inhibitors can restore sensitivity to tamoxifen [36]. Hence, factors whose activities are both estrogen-independent and sensitive to MAPK inhibitors likely contribute to some cases of tamoxifen resistance. ERRα1 meets these criteria: in ErbB2-over-expressing cells, the hyper-phosphorylated activator form of ERRα1 can functionally substitute for ER, likely rendering tamoxifen ineffective in inhibiting ERE-regulated transcription; blockage of the MAPK or Akt signaling pathways leads to conversion of ERRα1 to its repressor form, eliminating ERRα1's ability to substitute for ER as an activator and, thus, restoring tamoxifen sensitivity mediated though ER. Furthermore, the hypo-phosphorylated repressor form of ERRα1 down-modulates ER-stimulated transcription; hence, its presence may enhance the repressive effects of tamoxifen. Therefore, we conclude that ERRα1 likely plays a critical role in some cases of resistance to tamoxifen.

Another prediction of this model is that breast tumors expressing high levels of ErbB2 along with ERRα in its hyper-phosphorylated state likely will not respond well to hormonal-blockade therapies; rather, they may respond well to ErbB2-based therapies such as trastuzumab. Tumors also expressing EGFR may be suitable for gefitinib therapy. Thus, ERRα can be used as a biomarker for prognosis and predictor of outcome from specific therapeutic treatments. Moreover, ERRα has utility, itself, as a target for a new class of drugs, possibly used in combination with current therapies.

### EXAMPLE 2

### Generation by Standard Hybridoma Methods of a Panel of Monoclonal Antibodies (mAbs) to ERRα which Can be Used to Distinguish the Activator from the Repressor Form of this Nuclear Receptor

A panel of mouse mAbs can be generated by the hybridoma method as described in Harlow, E., and Lane, D. Antibodies: a laboratory manual. Cold Spring Harbor Laboratory Press. Cold Spring Harbor, New York, 1988, which is incorporated by reference in its entirety. ERRα protein can be generated in E. coli. E. coli is unlikely to add the desired post-translational modifications to the protein. Desired post-translational modifications (phosphorylation) can be added in vitro with the appropriate enzyme (e.g., MAPK). Alternatively, ERRα protein with desired posttranslational modifications can be obtained from ERRα-overexpressing HeLa cells or BT-474 cells. ERRα protein with and without the desired post-translational modifications are used as immunogens to generate a panel of mAbs. Alternatively, the post-translational modification(s) of ERRα crucial for activator functionality is identified by genetic or biochemical means. Synthetic peptide(s) containing this modification(s) is used as immunogen(s) to generate mAbs. The mAbs are screened for identification of antibodies that can react specifically with modified (phosphorylated) and unmodified ERRα proteins or activator and repressor forms of ERRα present in BT-474 and MCF-7 cells, respectively. These phospho-specific ERRα mAbs can then be used to assay for the hyper-phosphorylated, activated form of ERRα by immunoblots in a similar manner as activated MAPK and Akt were detected in FIG. 5D. Additionally, phospho-specific ERRα mAbs can be developed for use in immunohistochemical staining of either frozen or formalin-fixed, paraffin-embedded tissue sections of tumors by analogy to reference [5].

### EXAMPLE 3

### Differential Sensitivity to the Growth Inhibitory Effects of Trastuzumab and Gefitinib in BT-474 Cells Versus MCF-7 Cells

Method

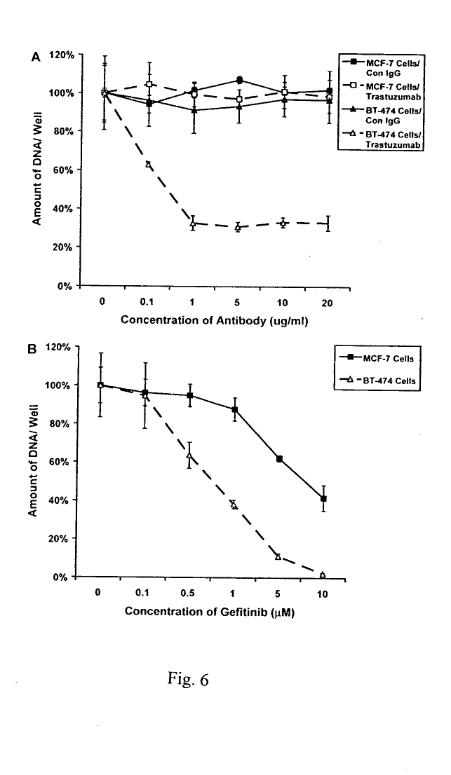

MCF-7 and BT-474 cells were seeded in 12-well plates at 15,000 and 30,000 cells per well, respectively. Cells were allowed to grow in the presence of the test agents for a total of 7 days, after which, they were harvested for measurement of DNA content per well using a fluorescent DNA quantification kit (Bio-Rad, Hercules, Calif.). Day 0 was considered to be 24 h after the cells were seeded, when the medium was initially replaced with fresh medium containing the test agents. The cell culture medium was again exchanged with fresh medium containing the appropriate test agents on days 2 and 5. The test agents were control murine IgG, trastuzumab and gefitinib, which were added to the cell culture medium at 1:1000 (v/v). The effects on cellular proliferation of the test agents are shown as the percentage of DNA amount per well relative to the control treated cells. The data are plotted as the mean ± SEM of three replicate wells (FIG. 6).

Results

As shown in FIG. 6, concentrations of 1 μg/ml to 20 μg/ml trastuzumab blocked the growth of BT-474 cells by about 67%, whereas parallel treatments with control, non-specific murine IgG had no effect on growth. In contrast to BT-474 cells, trastuzumab did not significantly block growth of MCF-7 cells (FIG. 6A). Similarly, at a concentration of 1I M gefitinib, BT-474 cells were growth inhibited by 62 %, whereas this concentration of gefitinib did not significantly affect the growth of MCF-7 cells (FIG. 6B).

### References

1. Nuclear Receptors Nomenclature Committee. A unified nomenclature system for the nuclear receptor superfamily. Cell, 97: 161-163, 1999.
2. Jensen, E. V. and Jordan, V. C. The estrogen receptor: a model for molecular medicine. Clin Cancer Res, 9: 1980-1989, 2003.
3. Giguere, V. To ERR in the estrogen pathway. Trends Endocrinol Metab, 13: 220-225, 2002.
4. Ariazi, E. A., Clark, G. M., and Mertz, J. E. Estrogen-related receptor alpha and estrogen-related receptor gamma associate with unfavorable and favorable biomarkers, respectively, in human breast cancer. Cancer Res, 62: 6510-6518, 2002.
5. Suzuki, T., Miki, Y., Moriya, T., Shimada, N., Ishida, T., Hirakawa, H., Ohuchi, N., and Sasano, H. Estrogen-Related Receptor {alpha} in Human Breast Carcinoma as a Potent Prognostic Factor. Cancer Res, 64: 4670-4676, 2004.
6. Xie, W., Hong, H., Yang, N. N., Lin, R. J., Simon, C. M., Stallcup, M. R., and Evans, R. M. Constitutive activation of transcription and binding of coactivator by estrogen-related receptors 1 and 2. Mol Endocrinol, 13: 2151-2162, 1999.
7. Zhang, Z. and Teng, C. T. Estrogen receptor-related receptor alpha 1 interacts with coactivator and constitutively activates the estrogen response elements of the human lactoferrin gene. J Biol Chem, 275: 20837-20846, 2000.
8. Chen, S., Zhou, D., Yang, C., and Sherman, M. Molecular basis for the constitutive activity of estrogen related receptor alpha-1. J Biol Chem, 276: 28465-28470, 2001.
9. Yang, N., Shigeta, H., Shi, H., and Teng, C. T. Estrogen-related receptor, hERR1, modulates estrogen receptor-mediated response of human lactoferrin gene promoter. J Biol Chem, 271: 5795-5804, 1996.
10. Vanacker, J. M., Delmarre, C., Guo, X., and Laudet, V. Activation of the osteopontin promoter by the orphan nuclear receptor estrogen receptor related alpha. Cell Growth Differ, 9: 1007-1014, 1998.
11. Lu, D., Kiriyama, Y., Lee, K. Y., and Giguere, V. Transcriptional regulation of the estrogen-inducible pS2 breast cancer marker gene by the ERR family of orphan nuclear receptors. Cancer Res, 61: 6755-6761, 2001.
12. Yang, C., Zhou, D., and Chen, S. Modulation of aromatase expression in the breast tissue by ERR alpha-1 orphan receptor. Cancer Res, 58: 5695-5700, 1998.
13. Kraus, R. J., Ariazi, E. A., Farrell, M. L., and Mertz, J. E. Estrogen-related receptor alpha 1 actively antagonizes estrogen receptor-regulated transcription in MCF-7 mammary cells. J Biol Chem, Vol. 277, pp. 24826-24834, 2002.
14. Grant, S., Qiao, L., and Dent, P. Roles of ERBB family receptor tyrosine kinases, and downstream signaling pathways, in the control of cell growth and survival. Front Biosci, 7: d376-389, 2002.
15. Lannigan, D. A. Estrogen receptor phosphorylation. Steroids, 68: 1-9, 2003.
16. McClelland, R. A., Barrow, D., Madden, T. A., Dutkowski, C. M., Pamment, J., Knowlden, J. M., Gee, J. M., and Nicholson, R. I. Enhanced epidermal growth factor receptor signaling in MCF7 breast cancer cells after long-term culture in the presence of the pure antiestrogen ICI 182,780 (Faslodex). Endocrinology, 142: 2776-2788., 2001.
17. Benz, C. C., Scott, G. K., Sarup, J. C., Johnson, R. M., Tripathy, D., Coronado, E., Shepard, H. M., and Osborne, C. K. Estrogen-dependent, tamoxifen-resistant tumorigenic growth of MCF-7 cells transfected with HER2/neu. Breast Cancer Res Treat, 24: 85-95, 1993.
18. Coutts, A. S. and Murphy, L. C. Elevated mitogen-activated protein kinase activity in estrogen-nonresponsive human breast cancer cells. Cancer Res, 58: 4071-4074, 1998.
19. Wright, C., Nicholson, S., Angus, B., Sainsbury, J. R., Famdon, J., Cairns, J., Harris, A. L., and Home, C. H. Relationship between c-erbB-2 protein product expression and response to endocrine therapy in advanced breast cancer. Br J Cancer, 65: 118-121, 1992.
20. Houston, S. J., Plunkett, T. A., Barnes, D. M., Smith, P., Rubens, R. D., and Miles, D. W. Overexpression of c-erbB2 is an independent marker of resistance to endocrine therapy in advanced breast cancer. Br J Cancer, 79: 1220-1226, 1999.
21. Dowsett, M., Harper-Wynne, C., Boeddinghaus, I., Salter, J., Hills, M., Dixon, M., Ebbs, S., Gui, G., Sacks, N., and Smith, I. HER-2 amplification impedes the antiproliferative effects of hormone therapy in estrogen receptor-positive primary breast cancer. Cancer Res, 61: 8452-8458, 2001.
22. Sladek, R., Bader, J. A., and Giguere, V. The orphan nuclear receptor estrogen-related receptor alpha is a transcriptional regulator of the human medium-chain acyl coenzyme A dehydrogenase gene. Mol Cell Biol, 17: 5400-5409, 1997.
23. Kraus, M. H., Popescu, N. C., Amsbaugh, S. C., and King, C. R. Overexpression of the EGF receptor-related proto-oncogene erbB-2 in human mammary tumor cell lines by different molecular mechanisms. Embo J, 6: 605-610, 1987.
24. Willy, P. J., Murray, I. R., Qian, J., Busch, B. B., Stevens, W. C., Jr., Martin, R., Mohan, R., Zhou, S., Ordentlich, P., Wei, P., Sapp, D. W., Horlick, R. A., Heyman, R. A., and Schulman, I. G. Regulation of PPARgamma coactivator 1alpha (PGC-1alpha) signaling by an estrogen-related receptor alpha (ERRalpha) ligand. Proc Natl Acad Sci U S A, 101: 8912-8917, 2004.
25. Leyland-Jones, B. Trastuzumab: hopes and realities. Lancet Oncol, 3: 137-144., 2002.
26. Moulder, S. L., Yakes, F. M., Muthuswamy, S. K., Bianco, R., Simpson, J. F., and Arteaga, C. L. Epidermal growth factor receptor (HER1) tyrosine kinase inhibitor ZD1839 (Iressa) inhibits HER2/neu (erbB2)-overexpressing breast cancer cells in vitro and in vivo. Cancer Res, 61: 8887-8895, 2001.
27. Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., Copeland, R. A., Magolda, R. L., Scherle, P. A., and Trzaskos, J. M. Identification of a novel inhibitor of mitogen-activated protein kinase kinase. J Biol Chem, 273: 18623-18632., 1998.
28. Vlahos, C. J., Matter, W. F., Hui, K. Y., and Brown, R. F. A specific inhibitor of phosphatidylinositol 3-kinase, 2-(4-morpholinyl)-8-phenyl-4H-1-benzopyran-4-one (LY294002). J Biol Chem, 269: 5241-5248, 1994.
29. Rogatsky, I., Zarember, K. A., and Yamamoto, K. R. Factor recruitment and TIF2/GRIP1 corepressor activity at a collagenase-3 response element that mediates regulation by phorbol esters and hormones. Embo J, 20: 6071-6083., 2001.
30. McKnight, S. L. and Kingsbury, R. Transcriptional control signals of a eukaryotic protein-coding gene. Science, 217: 316-324., 1982.
31. Jiang, S. Y., Wolf, D. M., Yingling, J. M., Chang, C., and Jordan, V. C. An estrogen receptor positive MCF-7 clone that is resistant to antiestrogens and estradiol. Mol Cell Endocrinol, 90: 77-86, 1992.
32. Katzenellenbogen, J. A., Johnson, H. J., Jr., and Myers, H. N. Photoaffinity labels for estrogen binding proteins of rat uterus. Biochemistry, 12: 4085-4092., 1973.
33. Johnston, S. D., Liu, X., Zuo, F., Eisenbraun, T. L., Wiley, S. R., Kraus, R. J., and Mertz, J. E. Estrogen-related receptor alpha 1 functionally binds as a monomer to extended half-site sequences including ones contained within estrogen-response elements. Mol Endocrinol, 11: 342-352, 1997.
34. O'Reilley, G. H. Regulation of the SV40 late promoter by nuclear receptors and large T antigen. Genetics. Madison: University of Wisconsin-Madison, 2000.
35. Knowlden, J. M., Hutcheson, I. R., Jones, H. E., Madden, T., Gee, J. M., Harper, M. E., Barrow, D., Wakeling, A. E., and Nicholson, R. I. Elevated levels of epidermal growth factor receptor/c-erbB2 heterodimers mediate an autocrine growth regulatory pathway in tamoxifen-resistant MCF-7 cells. Endocrinology, 144: 1032-1044, 2003.
36. Kurokawa, H., Lenferink, A. E., Simpson, J. F., Pisacane, P. I., Sliwkowski, M. X., Forbes, J. T., and Arteaga, C. L. Inhibition of HER2/neu (erbB-2) and mitogen-activated protein kinases enhances tamoxifen action against HER2-overexpressing, tamoxifen-resistant breast cancer cells. Cancer Res, 60: 5887-5894., 2000.

1. A method for determining whether a breast cancer patient is likely to respond to ErbB2-based therapy or hormonal-blockade therapy comprising the step of:
analyzing the phosphorylation status of ERRα in breast cancer cells of the patient wherein hyper-phosphorylation status of ERRα indicates that the patient is likely to respond to ErbB2-based therapy but not hormonal-blockade therapy.

2. The method of claim 1, wherein ErbB2-based therapy is a therapy based on targeting EGFR.

3. The method of claim 2, wherein the ErbB2-based therapy employs a small molecule tyrosine kinase inhibitor or a monoclonal antibody against EGFR.

4. The method of claim 3, wherein the small molecule tyrosine kinase inhibitor is gefitinib (ZD1839, Iressa).

5. The method of claim 1, wherein the ErbB2-based therapy employs an anti-HER2 antibody.

6. The method of claim 5, wherein the anti-HER2 antibody is trastuzumab (Herceptin).

7. A method for determining whether a breast cancer patient with breast cancer cells that express high levels of ErbB2 and ERRα is likely to respond to ErbB2-based therapy, the method comprising the step of:
analyzing the phosphorylation status of ERRα in breast cancer cells of the patient wherein hyper-phosphorylation status of ERRα indicates that the patient is likely to respond to ErbB2-based therapy.

8. The method of claim 7, wherein ErbB2-based therapy is a therapy based on targeting EGFR.

9. The method of claim 8, wherein the ErbB2-based therapy employs a small molecule tyrosine kinase inhibitor or a monoclonal antibody against EGFR.

10. The method of claim 9, wherein the small molecule tyrosine kinase inhibitor is gefitinib (ZD1839, Iressa).

11. The method of claim 7, wherein the ErbB2-based therapy employs an anti-HER2 antibody.

12. The method of claim 11, wherein the anti-HER2 antibody is trastuzumab (Herceptin).

13. A method for determining whether a breast cancer patient with breast cancer cells that express high levels of ERα and ERRα is insensitive to hormonal-blockade therapy by itself, the method comprising the step of:
analyzing the phosphorylation status of ERRα in breast cancer cells of the patient wherein hyper-phosphorylation status of ERRα indicates that the patient is insensitive to hormonal-blockade therapy by itself.

14. A method for treating breast cancer comprising the steps of:
analyzing the phosphorylation status of ERRα in breast cancer cells of a patient; and
reducing ERRα activity when hyper-phosphorylated ERRα is found in the patient's cancer cells.

15. The method of claim 14, wherein the ERRα activity is reduced by reducing the level of ERRα, reducing phosphorylation of ERRα, or exposing ERRα to an antagonist ligand.

16. The method of claim 14, wherein the ERRα phosphorylation is reduced by blocking the ErbB2 signaling pathway.

17. The method of claim 16, wherein the ErbB2 signaling pathway is blocked by blocking ErbB2 activity, EGFR activity, MEK (MAPK kinase) activity, MAPK activity, phosphatidylinositol-3-OH kinase (PI3K) activity, Akt activity or a combination of any of the foregoing.

18. A method of determining prognosis of a breast cancer patient comprising the step of analyzing the phosphorylation status of ERRα in breast cancer cells of the patient wherein hyper-phosphorylation status of ERRα indicates a poor prognosis and hypo-phosphorylation status of ERRα indicates a more favorable prognosis.

19. The method of claim 18, wherein the phosphorylation status of ERRα in breast cancers is determined by using antibodies which specifically recognize hyper-phosphorylated ERRα versus hypo-phosphorylated ERRα.

20. A method of identifying an agent that can modulate the phosphorylation of ERRα, the method comprising the steps of:
exposing a group of cells that express ERRα to a test agent; and
comparing the phosphorylation status of ERRα in the cells to that of control cells that are not exposed to the agent wherein a difference in the phosphorylation status of ERRα indicates that the agent can modulate the phosphorylation of ERRα and its activity.
